(19) United States
(12) Patent Application Publication
(10) Pub. No.: US 2024/0219395 A1
(43) Pub. Date: Jul. 4, 2024

(54) METHOD FOR DETERMINING FIBRINOGEN

(71) Applicant: Siemens Healthcare Diagnostics Products GmbH, Marburg (DE)

(72) Inventor: Juergen PATZKE, Marburg (DE)

(21) Appl. No.: 18/543,707

(22) Filed: Dec. 18, 2023

(30) Foreign Application Priority Data
Dec. 28, 2022 (EP) 22216869.2

(51) Int. Cl. G01N 33/68; G01N 33/86

(52) U.S. Cl. CPC G01N 33/68; G01N 33/86; G01N 2333/7454; G01N 2333/75; G01N 2333/755; G01N 2333/974

(57) ABSTRACT

The present invention is in the field of blood coagulation diagnostics and relates to an improved method for quantitatively determining fibrinogen in a sample. The method comprises adding factor XIII to the reaction mixture.

### PRIORITY STATEMENT

The present application claims priority under 35 U.S.C. § 119 to European Patent Application No. 22216869.2, filed 28 Dec. 2022, the entire contents of which are incorporated herein by reference.

### FIELD

The present invention is in the field of blood coagulation diagnostics and relates to an improved method for quantitatively determining fibrinogen in a sample.

### BACKGROUND

Fibrinogen is the water-soluble precursor of fibrin, which forms the matrix for wound closure. The coagulation protease thrombin (factor IIa) cleaves fibrinogen and in this way activates fibrin formation, i.e., clot formation. Lowered fibrinogen levels are associated with a tendency to bleed. Acutely elevated fibrinogen levels are frequently found in inflammation, after surgery and in other situations. Long-term elevated fibrinogen levels are considered to be a risk indicator for thrombotic disorders.

A number of different methods for determining fibrinogen concentration are known in the prior art.

CA 1062501 describes a fibrinogen determination method based on the measurement of thrombin time, it being known that thrombin time does not allow precise determination of fibrinogen concentration, since thrombin time can be influenced by not only fibrinogen but also other factors, for example anticoagulants such as heparin or direct thrombin inhibitors or else the presence of fibrin or fibrinogen cleavage products, and thrombin time is generally only effective in the case of severe fibrinogen-deficient states. A thrombin time method usually comprises mixing an undiluted plasma sample with a comparatively small amount of thrombin to activate coagulation and photometrically measuring fibrin formation, i.e., the change in absorbance of the reaction, and then determining the coagulation time. According to CA 1062501, it is, however, not coagulation time that is determined, but instead the maximum of the first derivative of the reaction curve or, in other words, the maximum change in absorbance of the reaction curve. It was found that the maximum change in absorbance correlates linearly with the fibrinogen concentration, and so the latter can be determined with the aid of a calibration curve constructed on the basis of assignment of known fibrinogen concentrations and maximum changes in absorbance.

A much more precise and commonly used method for determining fibrinogen concentration is the so-called Clauss method (Clauss, A., Gerinnungsphysiologische Schnellmethode zur Bestimmung des Fibrinogens [Rapid coagulation-based method for determining fibrinogen], 1957, Acta haemat. 17: 237-246). The test is a variation of thrombin time, comprising mixing a plasma sample with thrombin as coagulation activator and determining the coagulation time. In the Clauss method, a comparatively low fibrinogen concentration is combined with a comparatively high, standardized thrombin concentration in the reaction, as a result of which the rate of fibrin formation virtually solely correlates with the fibrinogen concentration. The comparatively low fibrinogen concentration in the reaction is usually established by the use of prediluted plasma samples.

Fibrin formation, i.e., clot formation, is then determined photometrically in the reaction. Owing to fibrin formation, the reaction becomes increasingly turbid, and so fibrin formation can be quantitatively measured by absorption measurement.

The coagulation time of the sample is then usually determined. The coagulation time of the sample is proportional to the amount of fibrinogen. The coagulation time of a sample is the time from when thrombin is added to the sample to when tangible fibrin formation, i.e., turbidity of the reaction, is measured. “Tangible fibrin formation” can be defined as a test- and instrument-specific threshold value which—if exceeded—indicates the coagulation time. Alternatively, “tangible fibrin formation”, proceeding from the signal difference before the start and after completion of the coagulation reaction, can be defined as a test- and instrument-specific percentage signal difference value which—if reached—indicates the coagulation time. The reaction kinetics of fibrin formation in a Clauss test differ considerably from the reaction kinetics of fibrin formation in a thrombin time test. In the Clauss test, fibrin formation starts as early as 3 seconds after addition of the thrombin reagent, depending on the fibrinogen concentration, i.e., much earlier than in the thrombin time test, in which fibrin formation does not start until after 10 seconds. In addition, in the Clauss test, the rate of fibrin formation is higher, at least in samples of comparatively high concentration, than in the thrombin time test. Therefore, the reaction curves for samples having a high fibrinogen concentration in the Clauss test are distinguished by a short lag phase, a steep slope and a relatively rapidly reached plateau phase compared to the thrombin time test. Compared to the thrombin time test, the reaction curves for samples having a low fibrinogen concentration in the Clauss test likewise show a short lag phase, a gentle slope, and a late plateau phase to the extent that it usually only starts after measurement has been completed. In the case of thrombin time by contrast, the plateau phase is much weaker or it is even completely inapplicable.

Another method for determining fibrinogen is the determination of so-called “derived fibrinogen”, i.e., the determination of fibrinogen derived from the determination of thromboplastin time. This is achieved by mixing a plasma sample with a thromboplastin (e.g., lipidated tissue factor) as coagulation activator and calculating the fibrinogen concentration (derived fibrinogen) from the fibrin formation curve measured by nephelometry or turbidimetry.

However, the known methods described, in particular the Clauss method, have the disadvantage that the measurement range is highly limited. Since the Clauss method requires contacting the sample with a comparatively high thrombin concentration so that the rate of fibrin formation virtually solely correlates with the fibrinogen concentration, this has the disadvantage that samples with just a slightly elevated fibrinogen content exhibit coagulation with an extremely rapid onset. For instance, samples having a fibrinogen content of about 5 g/L (normal range: 1.8-3.5 g/L) can have a coagulation time of just 3.8 seconds, a time span that is difficult to cover even for automated analysis systems. Samples having yet higher fibrinogen concentrations can only be analyzed reliably if they are diluted prior to measurement (e.g., 1:10 or 1:100 in buffer). However, if each sample were to be generally diluted prior to measurement so as to extend the measurement range in the upper fibrinogen concentration range, this would automatically limit the measurement range in the lower fibrinogen concentration range, since the amount of fibrinogen in the reaction in the case of samples with a reduced fibrinogen content is so little that insufficient or undetectable clot formation may occur. Therefore, it is typically necessary to measure many samples multiple times, since further measurements must be made at other sample dilutions after the first measurement. This approach is time-consuming and costly.

### SUMMARY

Therefore, a method for determining fibrinogen concentration that—compared to the known methods for determining fibrinogen that comprise providing a reaction mixture by mixing a typically diluted sample with at least one coagulation activator and measuring fibrin formation in the reaction mixture—covers an extended measurement range in a single measurement and hence distinctly reduces the number of necessary multiple measurements is provided herein.

### BRIEF DESCRIPTION OF THE FIGURES

FIG. 1A shows a calibration curve for determining fibrinogen concentration that was determined according to the prior art without addition of factor XIII to the reaction.

FIG. 1B shows a calibration curve for determining fibrinogen concentration that was determined according to the invention with addition of factor XIII to the reaction.

FIG. 2A shows the fibrinogen concentrations (g/L) of samples having a known fibrinogen concentration that were determined according to the prior art and determined without addition of factor XIII to the reaction.

FIG. 2B shows the fibrinogen concentrations (g/L) of samples having a known fibrinogen concentration that were determined according to the invention and determined with addition of factor XIII to the reaction.

### DETAILED DESCRIPTION

It has been found that adding factor XIII to the sample or to the reaction mixture composed of sample and coagulation activator extends the measurement range in the lower fibrinogen concentration range. This in turn makes it possible in many cases to dispense with multiple measurement of a sample having a reduced fibrinogen content.

Factor XIII is known as the enzyme that takes on the important role of crosslinking fibrin threads in clot formation. This crosslinking stabilizes the clot. In the plasma of healthy individuals, a factor XIII content of 70% to 140% is typically found.

Thus, in one embodiment the present invention provides a method for determining fibrinogen in a sample, comprising the following steps:
a) providing a reaction mixture by mixing the sample with at least one coagulation activator, and
b) measuring fibrin formation in the reaction mixture, wherein factor XIII is additionally added to the reaction mixture.

Preferably, there is added to the reaction mixture an amount of factor XIII such that the final concentration of added factor XIII in the reaction mixture corresponds to 5% to 200% of the norm. 100% of the norm corresponds to the factor XIII activity measured in a plasma pool from multiple healthy donors.

Isolated human factor XIII (purified from human plasma or produced synthetically) or recombinant factor XIII (obtained by gene technology), for example, can be added to the reaction mixture.

The factor XIII added can be added in the form of the (thrombin-)activatable proenzyme (factor XIII) or as an already activated enzyme (factor XIIIa).

Preferably, factor XIII is mixed with the sample before the coagulation activator is subsequently added to the reaction mixture. Alternatively, the factor XIII and the coagulation activator can be mixed with the sample at the same time, for example by adding a reagent containing factor XIII and the coagulation activator to the sample.

A suitable coagulation activator is any substance or mixture of substances that activates the extrinsic and/or intrinsic pathway of the blood coagulation system when added to a human plasma sample. Preferred coagulation activators are mixtures of tissue factor and phospholipids (thromboplastins) and calcium chloride. The tissue factor can originate from the brain, lungs or placental tissue of a mammal (e.g., human or rabbit), or it can alternatively be produced recombinantly or synthetically. Another preferred coagulation activator is thrombin, for example human or bovine thrombin. Alternatively, coagulation-activating snake venoms or a coagulation-activating protease isolated from snake venom can also be used as activator. Further coagulation activators are phospholipids and negatively charged surfaces such as glass, silica, kaolin, ellagic acid, celite and platelet factor 3.

The term “sample” is to be understood to mean a plasma or whole blood sample, preferably a human plasma or whole blood sample or animal plasma or whole blood sample. Plasma or whole blood samples can contain an anticoagulant such as citrate or EDTA, which is added during sample collection to avoid spontaneous, uncontrolled coagulation of the sample.

Measurement of fibrin formation in the reaction mixture is preferably carried out by measuring the absorbance values of the reaction mixture over time. Absorbance values can be measured by photometry, i.e., by measuring the light attenuation of a light beam transmitted through the reaction mixture, or by nephelometry, i.e., by measuring scattered light components of a light beam transmitted through the reaction mixture. Ideally, measurement is started immediately after the coagulation activator has been added to the reaction mixture, and absorbance values are measured continuously until fibrin formation has been completed.

Fibrin is subsequently quantitatively determined by evaluating the determined absorbance curve using an evaluation method familiar to a person skilled in the art, for example determining the maximum change in absorbance and comparing it with a calibration curve constructed on the basis of assignment of known fibrinogen concentrations and maximum changes in absorbance.

In another embodiment, the present invention further provides a reagent for use in a method for determining fibrinogen in a sample, the reagent containing at least one coagulation activator and factor XIII. The reagent allows particularly simple provision of a reaction mixture for determination of fibrinogen in accordance with the invention.

As already described above, the coagulation activator contained in the reagent can be any substance or mixture of substances that activates the extrinsic and/or intrinsic pathway of the blood coagulation system when added to a human plasma sample. Preferably, the reagent contains a coagulation activator from the group consisting of thrombin and thromboplastin.

As already described above, the factor XIII also contained in the reagent can be isolated human factor XIII or recombinant factor XIII and can be used either in the form of the activatable proenzyme (factor XIII) or as already activated enzyme (factor XIIIa).

In another embodiment, the present invention yet further provides a test kit for use in a method for determining fibrinogen in a sample. For this purpose, the test kit according to the invention comprises i) a first reagent containing at least one coagulation activator and ii) a second reagent containing factor XIII. The coagulation activator contained in the first reagent and the factor XIII contained in the second reagent can be embodied as described above in their respective embodiments.

### EXAMPLES

### Example 1: Clauss Fibrinogen Test in the Presence of Exogenous Factor XIII

The factor XIII concentrate Fibrogammin® 250 (CSL Behring, Marburg, Germany) was dissolved in an aqueous solution containing 9 g/L albumin, 8 g/L NaCl and 5 g/L glucose. The concentration of factor XIII in this solution was 7100% of the norm.

This factor XIII-containing solution was mixed with a reagent containing thrombin as coagulation activator (Dade Thrombin Reagent, Siemens Healthineers, Marburg, Germany). The above aqueous solution was also used to prepare a mixture without factor XIII. Table 1 shows the mixing ratios of the various components.

TABLE 1

| | | | | Factor XIII |
|---|---|---|---|---|
| | | Aqueous | | concentration |
| | Aqueous | solution | | in the |
| | solution | with factor | | thrombin |
| | without | XIII (7100% | Thrombin | reagent |
| | factor | of the norm) | reagent | mixture [% of |
| | XIII [μL] | [μL] | [μL] | the norm] |
| Without | 197.3 | 0 | 2802.7 | 0 |
| factor | | | | |
| XIII | | | | |
| With | 98.65 | 98.65 | 2802.7 | 233.5 |
| factor | | | | |
| XIII | | | | |

To determine fibrinogen in human plasma samples (standard human plasma), the reaction mixtures were prepared as follows:
24 μL of sample (prediluted 1:3 with Owren's Veronal Buffer)
10 seconds incubation at 37° C.
+76 μL of Owren's Veronal Buffer
180 seconds incubation at 37° C.
+50 μL of thrombin reagent mixture with/without factor XIII.

The concentration of factor XIII in the reaction mixtures thus prepared was 77.8% of the norm and 0% of the norm (without addition of factor XIII).

In the reaction mixture, absorption was measured continuously at 405 nm, and the reaction rate (mA/s) was determined in the range between 0 and 70 seconds.

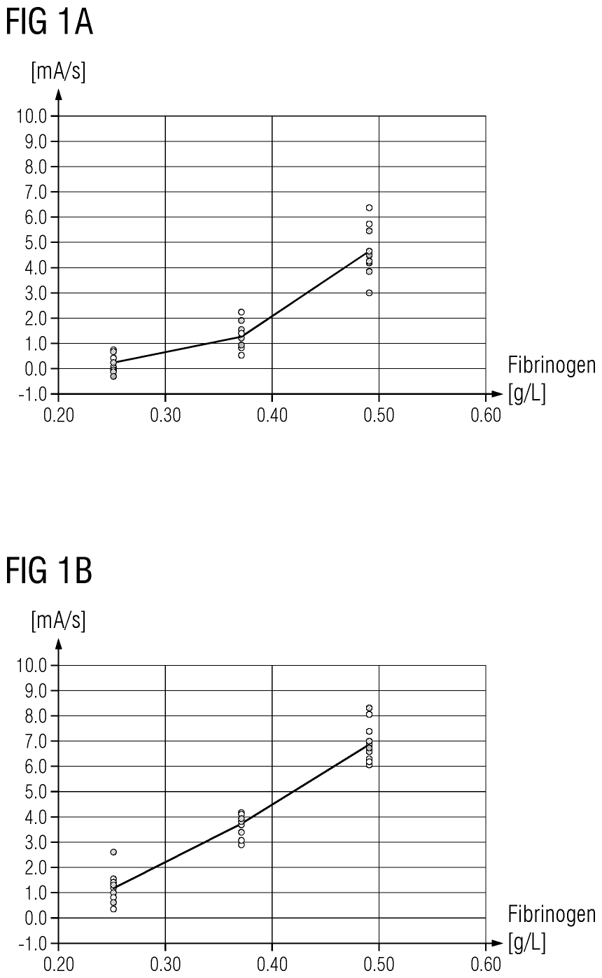

First of all, two calibration curves were determined. To this end, standard human plasma samples having fibrinogen concentrations of 0.25, 0.37 and 0.49 g/L that were diluted with Owren's Veronal Buffer were each measured 10 times in a fibrinogen determination test without addition of factor XIII (prior art, FIG. 1A) and in a fibrinogen determination test with addition of factor XIII (according to the invention, FIG. 1B). The concentration levels 0.25, 0.37 and 0.49 g/L fibrinogen were established taking into account the declared fibrinogen value in standard human plasma. The mean values from the 10-times determinations are used as supporting points for the calibration curves (FIGS. 1A and 1B).

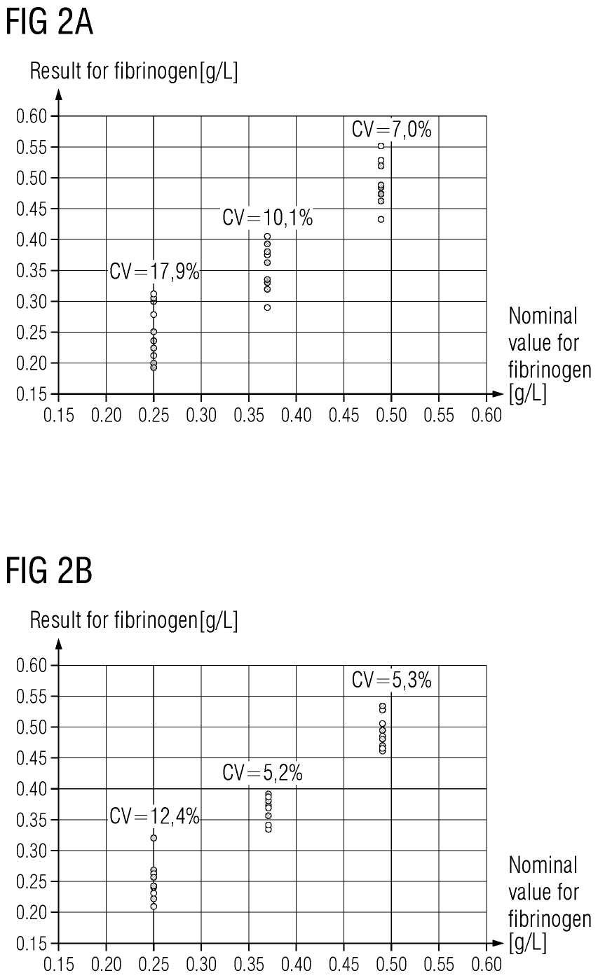

Thereafter, three plasma samples having known fibrinogen concentrations (0.25, 0.37 and 0.49 g/L) were each measured 10 times in a fibrinogen determination test without addition of factor XIII (prior art, FIG. 2A) and in a fibrinogen determination test with addition of factor XIII (according to the invention, FIG. 2B). The raw values were evaluated using the calibration curves from FIG. 1, and the coefficient of variation was determined for each 10-times determination.

From FIG. 1A, it can be seen that, without addition of factor XIII, the lower calibration point of 0.25 g/L fibrinogen is very close to the reaction rate of 0 mA/s. Individual values are in the negative range (falling absorbance). A positive value of 0.2 mA/s is yielded only when 10 determinations are averaged. It can be seen that a clear statistical differentiation of the calibration point at 0.25 g/L fibrinogen from that at 0.37 g/L fibrinogen is not possible, since the raw values have a range of overlap. If, by contrast, factor XIII is added, the level of the slope values (mA/s) rises, and the calibration point at 0.37 g/L is clearly differentiable from the calibration point at 0.25 g/L, since there is no longer a range of overlap (FIG. 1B).

FIG. 2 depicts the evaluation of the measured raw values using the calibration curves from FIG. 1. Without addition of factor XIII, the sample with the nominal value of 0.25 g/L fibrinogen has a wide range of scatter that overlaps with the range of scatter from the sample with the nominal value of 0.37 g/L fibrinogen (FIG. 2A). In the case of addition of factor XIII, the scatter of the values is lower, and the sample containing 0.25 g/L fibrinogen is clearly differentiable from the sample containing 0.37 g/L fibrinogen (FIG. 2B). The coefficients of variation are substantially lower with addition of factor XIII than without addition of factor XIII.

In this example, the addition of factor XIII brings about a lower end of the measurement range of 0.25 g/L fibrinogen, whereas without the addition of factor XIII, only a lower measurement range of 0.37 g/L fibrinogen would be possible. The addition of factor XIII to the reaction mixture thus allows extension of the measurement range, and so the method according to the invention can be used for reliable determination of the fibrinogen concentration in more samples having a low fibrinogen concentration.

What is claimed is:

1. A method for determining fibrinogen in a sample, the method comprising the steps of
a) providing a reaction mixture by mixing the sample with at least one coagulation activator, and
b) measuring fibrin formation in the reaction mixture,
wherein factor XIII is additionally added to the reaction mixture.

2. The method as claimed in claim 1, wherein there is added to the reaction mixture an amount of factor XIII such that the final concentration of added factor XIII in the reaction mixture corresponds to 5% to 200% of the norm.

3. The method as claimed in claim 1, wherein the coagulation activator is selected from the group consisting of thrombin and thromboplastin.

4. The method as claimed in claim 1, wherein the factor XIII additionally added is isolated human factor XIII or recombinant factor XIII.

5. The method as claimed in claim 1, wherein the factor XIII additionally added is activated factor XIIIa.

6. A reagent for use in a method for determining fibrinogen in a sample, the reagent containing at least one coagulation activator and factor XIII.

7. The reagent as claimed in claim 6, wherein the coagulation activator is selected from the group consisting of thrombin and thromboplastin.

8. The reagent as claimed in claim 6, wherein the factor XIII is isolated human factor XIII or recombinant factor XIII.

9. The reagent as claimed in claim 6, wherein the factor XIII is activated factor XIIIa.

10. A test kit for use in a method for determining fibrinogen in a sample, the test kit comprising
i) a first reagent containing at least one coagulation activator and
ii) a second reagent containing factor XIII.

11. The test kit as claimed in claim 10, wherein the first reagent contains a coagulation activator from the group consisting of thrombin and thromboplastin.

12. The test kit as claimed in claim 10, wherein the second reagent contains isolated human factor XIII or recombinant factor XIII.

13. The test kit as claimed in claim 10, wherein the factor XIII in the second reagent is activated factor XIIIa.
